(19) United States
(12) Patent Application Publication
(10) Pub. No.: US 2022/0378810 A1
(43) Pub. Date: Dec. 1, 2022

(54) ANIMAL FOOD COMPOSITION

(71) Applicant: MARS, INCORPORATED, McLean, VA (US)

(72) Inventor: Adrian WATSON, Aimargues (FR)

(73) Assignee: MARS, INCORPORATED, McLean, VA (US)

(21) Appl. No.: 17/621,369

(22) PCT Filed: Jul. 1, 2020
(86) PCT No.: PCT/US2020/040516

§ 371 Date: Dec. 21, 2021

(30) Foreign Application Priority Data
Jul. 2, 2019 (EP) 19183974.5

(51) Int. Cl. A61K 31/704; A23K 10/30; A23K 20/147; A23K 20/158; A23K 20/174; A23K 40/00; A23K 20/111; A23K 50/40; A23K 20/105; A23K 20/121; A61K 36/484; A61K 36/9066; A61K 31/12; A61K 31/201; A61K 31/185; A61K 31/047; A61K 31/355; A61P 17/04

(52) U.S. Cl. CPC A61K 31/704; A23K 10/30; A23K 20/147; A23K 20/158; A23K 20/174; A23K 40/00; A23K 20/111; A23K 50/40; A23K 20/105; A23K 20/121; A61K 36/484; A61K 36/9066; A61K 31/12; A61K 31/201; A61K 31/185; A61K 31/047; A61K 31/355; A61P 17/04

(57) ABSTRACT

The present disclosure provides an animal food composition comprising a source of glycyrrhizin, in combination with a source of curcuminoids, for use for preventing and/or treating allergic inflammatory skin diseases.

### CROSS-REFERENCE TO RELATED APPLICATIONS

This application claims priority to European patent application no. EP 19183974.5, filed on Jul. 2, 2019, which is incorporated herein by reference in its entirety.

### FIELD OF THE DISCLOSURE

The present disclosure relates to the field of animal food compositions for preventing and/or treating allergic inflammatory skin diseases.

### BACKGROUND OF THE DISCLOSURE

Allergic inflammatory skin diseases are chronic disorders characterized by clinical signs such as erythema, pain, redness, swelling, the appearance of small vesicles or papules on the skin. They are the second most common allergic skin disease in animals, in particular dogs, surpassed only by flea allergies. These allergic reactions can be brought on by typically harmless substances such as grass, mold spores, house dust mites, and other environmental allergens.

The primary symptoms of allergic inflammatory skin diseases are skin inflammation and pruritus usually associated with scratching and excoriation by the affected animal caused by an interaction between genetic and environmental factors. The allergic symptoms appear as eczematous skin and animals such as dogs with atopic dermatitis often suffer from pruritus, or severe itching, hair loss, excoriation of the skin from deep scratching, frequent licking of their paws and excessive tear production. Secondary skin problems such as skin infections and excessive sebum discharge are also common.

More particularly, atopic dermatitis is one of the most common allergic inflammatory skin diseases. Several treatments for atopic dermatitis and pruritus have already been described including the use of ultra-micronized palmitoylethanolamide (Noli et al.; Vet Dermatol 2015; 26, 432-40) or anti-histamines, such as fexofenadine, or drugs like cyclosporine. Nevertheless, most of the available medications are not suitable for long term use due to significant side-effects (as well as, in many cases, prohibitive cost). Although immunotherapy can improve signs if the allergens are identified, success rates remain variable. The ability to reduce the dependency on medications would be seen as a valuable contribution to the field and is the primary aim of nutritional interventions.

Indeed, there is evidence now that the genetic predisposition seems to influence the immune system as well as functional/structural aspects of the skin barrier. Known environmental components are allergen load and the microbial population present on the skin. Drugs currently prescribed for the condition target individual aspects of the course of the disease, typically immune related, but only control the disease rather than offering long term relief or offer a cure. A nutritional management strategy is more likely to be successful, if it targets multiple aspects simultaneously.

Thus, there remains a need for treatment options for allergic inflammatory skin diseases, such as atopic dermatitis.

There also remains a need for alternative natural products for treating or preventing such diseases.

Thus, there remains a need for novel diets designed to improve both immune function and barrier defences.

### SUMMARY

A first aspect of the present disclosure relates to an animal food composition comprising: (i) a source of glycyrrhizin, in combination with (ii) a source of one or more curcuminoids, for use in a method for preventing and/or treating allergic inflammatory skin diseases.

A second aspect of the present disclosure relates to a kit comprising (i) a first part comprising a source of glycyrrhizin and (ii) a second part comprising a source of one or more curcuminoids, for use in a method for preventing and/or treating allergic inflammatory skin diseases.

A third aspect of the present disclosure relates to an animal food composition comprising: (i) a source of glycyrrhizin, in combination with (ii) a source of one or more curcuminoids; wherein the animal food composition is a kibble and/or wherein the animal food composition comprises a source of proteins.

A fourth aspect of the present disclosure relates to a method of manufacturing an animal food composition comprising a source of glycyrrhizin, in combination with a source of one or more curcuminoids, the animal food composition being a kibble and/or the animal food composition comprising a source of proteins, said method comprising at least the steps of: a) mixing a source of glycyrrhizin and a source of one or more curcuminoids, thereby providing a mixture; and b) heating the mixture.

Another aspect of the present disclosure relates to the use of a source of glycyrrhizin and a source of one or more curcuminoids; for the preparation of a kit comprising a source of glycyrrhizin and a source of one or more curcuminoids; and for the preparation of an animal food composition comprising: (i) a source of glycyrrhizin, in combination with (ii) a source of one or more curcuminoids; wherein the animal food composition is a kibble and/or the animal food composition comprises a source of proteins.

In certain embodiments, the one or more curcuminoids present in the animal food composition or the kit disclosed herein is a compound of general formula (I), a pharmaceutically acceptable salt, a tautomer, or a stereoisomer thereof:

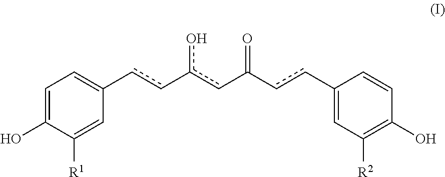

wherein R¹ and R² are independently hydrogen atom methoxy, methyl, hydroxyl or ethoxy group. In certain other embodiments, the one or more curcuminoids are selected from the group consisting of curcumin, demethoxycurcumin, methoxycurcumin, tetrahydrocurcumin, and combinations thereof.

In certain embodiments, the animal food composition disclosed herein is a nutritionally complete animal food composition.

In certain embodiments, the source of glycyrrhizin is the animal food composition of the present disclosure is licorice, and/or the source of the one or more curcuminoids in the animal food composition is turmeric.

In certain embodiments, the animal food composition disclosed herein further includes linoleic acid. In certain other embodiments, the animal food composition further includes EPA/DHA, Taurine, Lutein, Vitamin E or combinations thereof.

In certain embodiments, the animal food composition disclosed herein is used for treating and/or preventing an allergic inflammatory skin disease selected from the group consisting of atopic dermatitis, flea allergic dermatitis, urticaria, angioedema, inhalant allergy, inhalant allergic dermatitis, food allergic dermatitis, contact dermatitis, miliary dermatitis, eosinophilic granuloma, head and neck pruritus, and generalized pruritus.

In certain embodiments, the present disclosure is directed to an animal food composition including a source of glycyrrhizin, in combination with a source of one or more curcuminoids, wherein the animal food composition consists of a kibble and/or the animal food composition further comprises a source of proteins. In certain embodiments, the animal food composition is a nutritionally complete food composition. In certain embodiments, the source of glycyrrhizin in the animal food composition and/or the kit of the present disclosure is licorice and the source of one or more curcuminoids is turmeric.

In certain embodiments, the present disclosure provides a method for preventing and/or treating an allergic inflammatory skin disease in an animal in need thereof, wherein the method includes administering an animal food composition including a source of glycyrrhizin, and a source of one or more curcuminoids. In certain embodiments, the one or more curcuminoids is a compound of general formula (I), a pharmaceutically acceptable salt, a tautomer, or a stereoisomer thereof:

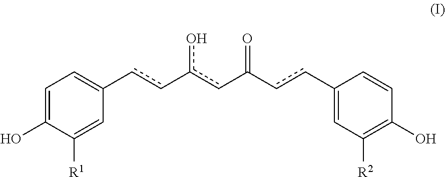

wherein R¹ and R² are independently hydrogen atom methoxy, methyl, hydroxyl or ethoxy group. In certain particular embodiments, the one or more curcuminoids are selected from the group consisting of curcumin, demethoxycurcumin, methoxycurcumin, tetrahydrocurcumin, and combinations thereof. In certain embodiments, the source of glycyrrhizin is licorice. In certain particular embodiments, the animal food composition includes licorice and turmeric. In certain embodiments, the animal food composition further includes linoleic acid, EPA/DHA, Taurine, Lutein, Vitamin E or combinations thereof.

In certain embodiments, the allergic inflammatory skin disease is selected from the group consisting of atopic dermatitis, flea allergic dermatitis, urticaria, angioedema, inhalant allergy, inhalant allergic dermatitis, food allergic dermatitis, contact dermatitis, miliary dermatitis, eosinophilic granuloma, head and neck pruritus, and generalized pruritus. In certain embodiments, the animal is a canine. In certain particular embodiments, the canine is a dog.

### DETAILED DESCRIPTION

The present disclosure aims at making available a product for preventing and/or treating allergic inflammatory skin diseases, especially atopic dermatitis, in animals, especially in dogs, i.e., canine atopic dermatitis.

The present disclosure provides herein an animal food composition comprising a source of glycyrrhizin, in combination with a source of curcuminoids.

Surprisingly, it has been shown that this animal food composition is capable of acting on the progression of pruritus, erythema, pain, redness, swelling, small vesicles or papules in the animal. The efficiency of this composition has been established based on through 3 outcomes: CADESI-04, PVAS, and Drug Score.

As it is shown in the Examples herein, the animal food composition of the present disclosure, when provided to animals with allergic inflammatory skin diseases, such as animals with atopic dermatitis, e.g., canine atopic dermatitis, is well tolerated and significantly impacts CADESI-04, PVAS and Drug Score at 3 months onwards and especially the animal food composition also had a significant effect on PVAS within the first month of the treatment.

In certain embodiments, as shown in the present disclosure, the combination of a source of glycyrrhizin and a source of one or more curcuminoids provides a benefit in terms of skin health of an animal, such as an animal affected with an allergic inflammatory skin disease. In certain particular embodiments, the animal is affected with an atopic dermatitis, e.g., a canine atopic dermatitis.

In certain other embodiments, as shown in the present disclosure, the combination of the above ingredients provides a benefit in terms of skin health of an animal. In particular embodiments, the animal is affected with an allergic inflammatory skin disease. In certain particular embodiments, the animal is affected with an atopic dermatitis, e.g., a canine atopic dermatitis.

Furthermore, as shown in the present disclosure, the combination of the above ingredients significantly reduces the severity and frequency of pruritic flares, which are the primary symptom of the disease. Specifically, the diet is shown to significantly reduce the dose and frequency of drugs required to manage the condition, i.e., reduce the severity of the condition to a level considered acceptable to vet and owner.

Hence, the present disclosure relates to an animal food composition or a kit comprising a source of glycyrrhizin, in combination with a source of curcuminoids. Such a composition or kit is useful for preventing and/or treating allergic inflammatory skin diseases in animal, and more particularly an animal affected with atopic dermatitis. In certain embodiments, the said animal is a canine. In certain particular embodiments, the said animal is a dog.

More particularly, the present disclosure also provides an animal food composition. This goal has been reached by providing an animal food composition, comprising a source of glycyrrhizin and a source of curcuminoids; characterized in that it is present in the form of a kibble, and/or it further comprises a source of proteins.

### Definitions

The terms used in this specification generally have their ordinary meanings in the art, within the context of this disclosure and in the specific context where each term is used. Certain terms are discussed below, or elsewhere in the specification, to provide additional guidance in describing the compositions and methods of the disclosure and how to make and use them.

As used in the specification and the appended claims, the singular forms “a,” “an” and “the” include plural referents unless the context clearly dictates otherwise. Thus, for example, reference to “a compound” includes mixtures of compounds.

The term “about” or “approximately”, as used herein, means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 10%, more preferably up to 5%, and more preferably still up to 1% of a given value.

As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

In the detailed description herein, references to “embodiment,” “an embodiment,” “one embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

As used herein, the term “enantiomers” refers to a pair of stereoisomers that are non-superimposable mirror images of each other. A 1:1 mixture of a pair of enantiomers is a “racemic” mixture or a racemate. The term is used to designate a racemic mixture where appropriate.

As used herein, the term “enantiopure” refers to a sample that within the limits of detection consists of a single enantiomer.

As used herein, the term “diastereoisomers” refers to stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro or levorotatory) in which they rotate plane polarized light at the wavelength of the sodium D line. The compounds of the presently disclosed subject matter contain one or more asymmetric centers and can thus give rise to enantiomers, diastereomers, and other stereoisomeric forms that can be defined, in terms of absolute stereochemistry, as (R)- or (S)-. The presently disclosed subject matter is meant to include all such possible isomers, including racemic mixtures, optically pure forms, and intermediate mixtures. Optically active (R)- and (S)-isomers can be prepared using chiral synthons or chiral reagents or resolved using conventional techniques. If the compound contains a double bond, the substituent can be E or Z configuration. If the compound contains a disubstituted cycloalkyl, the cycloalkyl substituent can have a cis- or trans-configuration. All tautomeric forms are also intended to be included.

As used herein, the term “isomers” refers to different compounds that have the same molecular formula but differ in arrangement and configuration of the atoms. Also, as used herein, the term “stereoisomer” refers to any of the various stereo isomeric configurations which can exist for a given compound of the presently disclosed subject matter and includes geometric isomers. It is understood that a substituent can be attached at a chiral center of a carbon atom. Therefore, the term stereoisomer includes enantiomers, diastereomers, or racemates of the compound. Also, as used herein, the terms “constitutional isomers” refers to different compounds which have the same numbers of, and types of, atoms but the atoms are connected differently, such as, but not limited to tautomers.

As used herein, the term “food composition” or “diet” covers all of foodstuff, diet, food supplement or a material that can contain proteins, carbohydrates and/or crude fats. Foods can also contain supplementary substances or additives, for example, minerals, vitamins and condiments (See Merriam-Webster's Collegiate Dictionary, 10th Edition, 1993). Such food compositions can be nutritionally complete or not. In a particular embodiment, an animal food composition according to the present disclosure is a nutritionally complete food composition.

As used herein, “nutritionally complete” means that the composition provides the complete and balanced nutritional requirement to animals. In certain embodiments such animals are dogs. Therefore, a nutritionally adequate feed is a feed with which the said animal, e.g., the said dog, can be fed as the sole ration and is capable of sustaining life without additional food (except water). The food composition can contain a carrier, a diluent, or an excipient. Depending on the intended use, the carrier, diluent, or excipient can be chosen to be suitable for animal use. In certain embodiments, the animal is a canine. In certain particular embodiments, the animal is a dog. In a general manner, nutritionally complete compositions comprise at least one source of proteins (or polypeptides), such as protein extracts, at least one source of vitamins, minerals, trace elements and fats.

As used herein, a “food supplement” refers to a concentrated source of nutrients, such as but not limited to vitamins and minerals, substances for nutritional or physiological purposes, or plants and plant preparations intended to compensate for deficiencies in an animal's regular diet. In certain embodiments, a food supplement is marketed in the form of capsules, lozenges, tablets, pills, powder packets or liquid forms (ampoules, vials with droppers).

As used herein, the term “drug” refers to any substance or composition represented as having treatment, curative or prevention properties against animal diseases. By extension, a medicinal product includes any substance or composition that can be used or administered to animals for the purpose of making a medical diagnosis or restoring, correcting or modifying their physiological functions by exercising a pharmacological, immunological or metabolic action.

Illustratively, a food composition as described herein can include, but it is not limited to protein, crude fat, ash, crude fiber, starch, calcium, phosphorus, sodium, chloride, potassium, magnesium, iron, water, copper, manganese, zinc, selenium, vitamin A, vitamin D3, vitamin B1, vitamin B2, vitamin B6, vitamin B12, vitamin B7, vitamin B9, cholin chloride, arachidonic acid, W3 fatty acid or W6 fatty acid.

As used herein, the term “animal” or “animals” designates a ruminant, poultry, swine, mammal, horse, mouse, rat, rabbit, guinea pig, hamster, cow, cat or canine. In particular embodiments, the animal is a canine. The term animal can refer more specifically to non-human animals. It may refer to domestic or wild animals. In certain embodiments, a non-human animal can be a pet animal. In certain embodiments, the pet animal is a cat or a canine. In certain particular embodiments, the canine is a dog.

For example, but not by the way of limitation, the animal can be a penguin, falcon, agouti, american kestrel, snake, bear, condor, ant, impala, antelope, armadillo, australian brush-turkey, babirusa, bald eagle, bali myna, fox, bat, sunbird, bee, beetle, bear cat, binturong, bird of paradise, boa, bonobo, bontebok, brown bear, caecilian, camel, caracal, chameleon, cheetah, chimpanzee, chinchilla, cichlid, clouded leopard, cobra, cockatoo, crocodilian, eagle, dhole, dolphin, platypus, echidna, elephant, emu, flamingo, fossa, frog, toad, galapagos, gelada, giant anteater, giant panda, gila monster, giraffe, goat, sheep, frog, gorilla, gouldian finch, guam rail, guanaco, guenon, hippo, hornbill, ibis, iguana, jacana, jaguar, kagu, kangaroo, wallaby, kingfisher, kingsnake, kinkajou, kiwi, klipspringer, koala, koi, komodo dragon, ladybug, laughing kookaburra, lemur, leopard, lion, lizard, lynx, macaw, magpie goose, manatee, mandrill, mangabey, marsupial, meerkat, metallic starling, monkey, ocelot, okapi, opossum, orangutan, oryx, ostrich, otter, owl, painted dog, parrot, peafowl, pelican, polar bear, porcupine, prairie dog, pronghorn, przewalski's horse, raccoon, radjah shelduck, red panda, reindeer, rhinoceros, ringtail, rock hyrax, saiga, scorpion, sea lion, secretary bird, serval, siamang, snow leopard, spider, stork, sun bear, surinam toad, takin, tapir, tasmanian devil, tenrec, tiger, toucan, tuatara, turtle, vulture, warthog, waxbill, whale, cape buffalo, wolf, wombat or zebra.

As used herein, the term “canine” encompasses animals, including pet animals, selected from the group consisting of wolf, coyote, jackal, dingo, dhole, fox, raccoon dog and dogs. As used herein, dogs encompass wild dogs and domestic dogs. In certain particular embodiments, the dogs are domestic dogs.

As used herein, the term “preventing”, can also include the reduction of a likelihood of occurrence, or of re-occurrence, of a given condition in an animal.

As previously mentioned herein, allergic inflammatory skin disease is characterized by producing one or more of erythema, pain, redness, swelling and small vesicles or papules on the skin. Body regions that are frequently affected in atopic dermatitis include the head, pinnae, feet, ventral abdomen and axillae. As used herein, the term “allergic” refers to a deregulated or unregulated sensitivity, in particular a hypersensitivity, of the immune system to typically harmless substances, such as those which are present in the environment. Common allergy triggers include but are not limited to airborne allergens, such as pollen, animal dander, dust mites and mold; certain foods, such as peanuts, tree nuts, wheat, soy, fish, shellfish, eggs and milk; insect stings, such as but not limited to from a bee or wasp; medications, such as penicillin or penicillin-based antibiotics; latex or other substances which can cause allergic skin reactions; allergic inflammatory skin disease such as atopic dermatitis, flea allergic dermatitis, urticaria, angioedema, inhalant allergy, inhalant allergic dermatitis, food allergic dermatitis, contact dermatitis, miliary dermatitis, eosinophilic granuloma, head and neck pruritus and generalized pruritus. It will thus be understood herein that the term “allergic” can relate both to “auto-antigens” and to exogenous antigens.

As used herein, the term “inflammation” refers to a local response to cellular injury that is marked by capillary dilatation, leukocytic infiltration, redness, heat and pain.

It is known by the skilled person in the art that allergic inflammatory skin disease, can be diagnosed by several ways. The allergic inflammatory skin disease can be diagnosed by measuring 3 outcomes. As used herein “3 outcomes” refers to CADESI-04, PVAS (Pruritus) and Drug Score.

As used herein, the term “CADESI-04” refers to “Canine Atopic Dermatitis Extent and Severity Index” (Olivry et al., 2014; International Committe on Allergic Diseases of Animals (ICADA)), one of the scoring systems developed to diagnose severity of canine atopic dermatitis. CADESI-04 can also be used alongside the Canine Atopic Dermatitis Lesion Index to assess degree of erythema, lichenification and excoration. CADESI-04 is a stand-alone scoring system to assess the likelihood that an animal has allergic inflammatory skin diseases, especially atopic dermatitis, canine atopic dermatitis and assess the severity of the condition.

Pruritus Visual Analogue Score (PVAS) is measured with the Hills' Pruritus Visual Analogue Score (Hill et al., 2007). PVAS is scored by the scorer using a zero to ten analog scale, with a score of zero representing no pruritus/chewing and a score of ten equating to incessant and intense pruritus/chewing.

Drug Score or Medication Score was based on the system developed and used by Litzbauer and co-workers (Litzlbauer P, et al., 2014; Oral and subcutaneous therapy of canine atopic dermatitis with recombinant feline interferon omega). Drug Score allows to determine and quantify the degree of dependence of an allergic inflammatory skin disease in animals, such as atopic dermatitis in animals, e.g., atopic dermatitis in dogs on drugs or other medications in order to maintain quality of life at a level acceptable to the owner and vet. Points were assigned to each medication (Tables 1A-1D). Tables 1A-D present the Drug Score used for animals with atopic dermatitis and concurrent medications.

TABLE 1A

| Medications: | Scores |
|---|---|
| No concurrent medication | 0 |
| Shampoo Therapy | 5 |
| Ear medication (topical) | 5 |
| Other topical Therapy | 5 |
| Antihistamins | 10 |
| Frequent antibiotics (>21 days) | 20 |
| Less frequent (<21 days) | 10 |

TABLE 1B

| Prednisolon: | Scores |
|---|---|
| ≥1 mg/kg/d | 40 |
| 0.5 mg/kg/d-1 mg/kg/d | 30 |
| 0.2 mg/kg/d-0.5 mg/kg/d | 20 |
| ≤0.2 mg/kg/d | 10 |

TABLE 1C

| Cyclosporin: (5 mg/kg) | Scores |
|---|---|
| SID (Once Daily) | 30 |
| EOD (Every other day) | 20 |
| E3D (Every third day) | 10 |
| E4D (Every fourth day) | 5 |

TABLE 1D

| Oclacitinib: | Scores |
|---|---|
| BID (Twice per day) | 40 |
| SID (Once daily) | 30 |
| EOD (Every other day) | 20 |
| E3D (Every third day) | 10 |

As used herein, the term “wet food” or “wet food composition” generally refers to a food composition having a moisture content of about 30% or more, generally of more than about 40% by weight, relative to the total weight of the food composition. In certain particular embodiments, the wet food composition has a moisture content lower than about 90% in weight, relative to the total weight of the food composition. In general, it is the final product of a process comprising a final step of sterilization (instead of a drying step). In a certain embodiments, the wet food consists of a chunk form, more particularly of chunks in gravy form. In certain particular embodiments, the wet food consists of chunks and gravy, chunks in jelly, loaf, mousse, terrine, bites form.

“Chunks and gravy” products comprise a preformed meat particle prepared by making a meat emulsion and by putting this meat emulsion through a muzzle under pressure and then cooked. A product, such as cooked meat, is diced into chunks, which are eventually mixed with a gravy or sauce. The two components are then filled into a container, usually a can or pouch, which is seamed or sealed and sterilized. As opposed to the ground loaf, chunk and gravy compositions have physically separated, discrete chunks (i.e., pieces of ground meat and grains) as prepared. These discrete particles are present in the gravy-type liquid in the final container. When serving, chunk and gravy products flow out of the can and can be easily mixed with other dry products. While the chunk and gravy products allow better integrity of the individual ingredients, the heterogeneous formulation of the chunk and gravy products are sometimes disfavored by consumers.

Wet food compositions are generally packaged in can-like containers and are considered “wet” in appearance because of the moisture contained therein. Two types of wet compositions are generally known in the art. The first is known in the art as “ground loaf.” Loaf products are typically prepared by contacting a mixture of components under heat to produce an essentially homogeneous, intracellular honeycomb-type mass or “ground loaf” The ground loaf mass is then packaged into a cylindrical container, such as a can. Upon packing, ground loaf assumes the shape of the container such that the ground loaf must be cut when serving to a companion animal.

In certain embodiments, the wet food composition is packaged. In this way, the consumer is able to identify, from the packaging, the ingredients in the food product and confirm that it is suitable for the particular pet in question. The packaging can be metal, plastic, paper or card.

As used herein, the term “dry food” or “dry food composition” generally refers to a food or composition having a moisture content of less than about 12% by weight, relative to the total weight of the food composition, and commonly even less than about 7% by weight, relative to the total weight of the food composition. In certain particular embodiments, dry food according to the present disclosure has a moisture content of at most about 12% by weight. In certain embodiments, the said dry food has a moisture content of about 7% or less, such as about 5% by weight. In certain particular embodiments, the dry food has a moisture content of more than about 3% by weight, relative to the total weight of the food composition. For instance, the Examples provided herein illustrate a dry food having a moisture content of about 9.5% by weight, relative to the total weight of the food composition. In a certain particular embodiment, the dry food consists of a kibble. In certain embodiments, for example and without limitation, kibbles can include particulates; pellets; pieces of pet food, dehydrated meat, meat analog, vegetables, and combinations thereof; and pet snacks, such as meat or vegetable jerky, rawhide, and biscuits. The dry food composition can be manufactured by mixing together ingredients and kneading in order to make consistent dough that can be cooked. In general, it can be the final product of a process comprising an extrusion step followed by a drying step.

The process of creating a dry food is usually done by baking and/or extruding. The dough is typically fed into a machine called an expander and/or extruder, which uses pressurized steam or hot water to cook the ingredients. While inside the extruder, the dough is under extreme pressure and high temperatures. The dough is then pushed through a die (specifically sized and shaped hole) and then cut off using a knife. The puffed dough pieces are made into kibble by passing it through a dryer so that moisture is dropped down to a defined target ensuring stability of the food until consumption. The kibble can then be sprayed with fats, oils, minerals, vitamins, the natural extracts cocktail and optionally sealed into packages. In certain particular embodiments, the dry food composition is packaged. In this way, the consumer is able to identify, from the packaging, the ingredients in the food product and confirm that it is suitable for the particular pet, e.g., dog, in question. The packaging can be metal, plastic, paper or card.

As used herein, the term “semi-moist food” or “semi-moist food composition” generally refers to a food composition with an intermediate moisture content of about 12% to about 30% in weight, relative to the total weight of the food composition. Hence, such semi-moist food composition is generally the final product of a process allowing a moisture content value that is intermediate between a dry food and a wet food. In certain embodiments, the said process can comprise a step of adding a humectant agent. In certain embodiments, the said process comprises an extrusion step and a subsequent treatment step with Super-Heated Stream (SHS). In certain embodiments, the semi-moist food according to the present disclosure contains more than about 12% and at most about 30% moisture by weight, relative to the total weight of the food composition. In certain embodiments, a semi-moist food composition has about 11% to about 20% moisture by weight, relative to the total weight of the food composition, and/or a water activity of about 0.64 to about 0.75. In certain particular embodiments, a semi-moist food composition has about 11% to about 20% moisture by weight, relative to the total weight of the food composition, and a water activity of about 0.64 to about 0.75.

As used herein, the term “water activity” refers to the ratio of the partial vapor pressure of water in a food composition divided by the standard state partial vapor pressure of water. Several methods, known to the skilled person, can be employed to measure the water activity such as but not limited to a resistive electrolytic, a capacitance or a dew point hygrometer. In certain particular embodiments, it is referred to the method prescribed by the International Standard ISO 21087 relating to determining water activity in animal food and animal feeding stuffs.

As non-limitative example, semi-moist food can be obtained using Super-Heated Stream (SHS) processes such as processes or methods described in the published patent applications WO2009/018990, WO2009/018996, WO2010/112097, WO2014/122072, WO2016/071372 and/or WO2016/071367, the disclosures of which are incorporated herein in their entireties.

In a certain particular embodiment, when the animal food composition is a semi-moist food, it consists of soft semi-moist kibbles.

According to certain embodiments, the moisture content of an animal food composition can be determined by a Loss on Drying Method, comprising the steps of:

(a) Weighing a sample of a food composition, thereby obtained the total weight;

(b) Heating, such as 135±2° C. for 240 minutes, the sample of step a) in an oven until the moisture is all driven off, thereby obtained a dry sample;

(c) Weighing the dry sample; thereby obtained the dry weight;

(d) Calculating the difference between the total weight and the dry weight, thereby obtaining the moisture weight, as:

Moisture weight=(total weight)−(dry weight)

### Units

All weight percentages expressed herein are by weight of the total weight of dry matter of the food composition unless expressed otherwise.

As used herein, an amount of a component as expressed as weight/Mcal consists of a weight amount of the said component by unit of Metabolizable Energy (ME) of the total animal food composition.

As used herein, the ME parameter is intended to represent the energy value of a food composition that is directly metabolized after consumption. Within the scope of the present disclosure, the ME value can be measured by any suitable method known in the art.

Illustratively, the ME value can be measured using feeding trial. In practice, the gross energy (GE) of the food is determined in the laboratory, and the amounts of food eaten by the animals are recorded. The feces and urine from the animals are collected, and the energy in each is determined and called fecal energy (FE) and urinary energy (UE), respectively. The ME is then calculated as:

ME (kcal/kg)=[GE−(FE+UE)]/Kg of food consumed.

Alternatively, the ME value can be measured by a mathematical method, in particular taking into account the percentage of crude fat (CF), of crude protein (CP), and NFE (carbohydrates) in the composition. In practice each percentage is multiplied by its respective Atwater Factors. The resulting sum is then multiplied by 10. The mathematical method can be represented by the following formula:

ME (kcal/kg)=10×[(3.5×CP)+(8.5×CF)+(3.5×NFE)].

Metabolizable Energy is conventionally determinable according to standard methods, and especially according to the NRC, nutrient requirements of dogs and cats, 2006, academy press, Washington D.C.

### Components of Food Compositions

The food compositions and kits of the present disclosure can comprise a wide variety of components. Non-limiting examples of components that can be incorporated in the food compositions of the present disclosure are listed below.

### Glycyrrhizin

In certain embodiments, the food compositions and/or kits described herein can include glycyrrhizin. As used herein, the term “glycyrrhizin” designates a constituent of Glycyrrhiza glabra root, i.e., licorice or liquorice. Glycyrrhizin, the most prominent compound found in licorice, is a triterpene glycoside. Licorice is a perennial plant naturally occurring or cultivated in Europe and Asia. Further, glycyrrhizin has been attributed numerous pharmacological effects like anti-inflammatory, anti-viral, anti-tumor, antioxidant and hepatoprotective activities (Sato et al., 1996; Rahman et al., 2006).

The structure of glycyrrhizin encompasses a triterpene portion (glycyrrhetinic acid) and two iduronic acid residues. Other names for glycyrrhizin include Glycyrrhizinic acid, Glycyrrhizic acid, Glycyrrhetinic acid glycoside, and (3-beta,20-beta)-20-Carboxy-11-oxo-30-norolean-12-en-3-yl-2-O-beta-D-glucopyranuronosyl-alpha-D glucopyranosiduronic acid.

Glycyrrhizin can be extracted from various natural plants by several extraction techniques known by a skilled person in the art, such as, for example, supercritical CO₂ extraction, aqueous two-phase extraction, solvent extraction, third-phase extraction, microwave-assisted extraction or ultrasound-assisted extraction.

Glycyrrhizin has CAS Registry Number: 1405-86-3 and is characterized by the following structure:

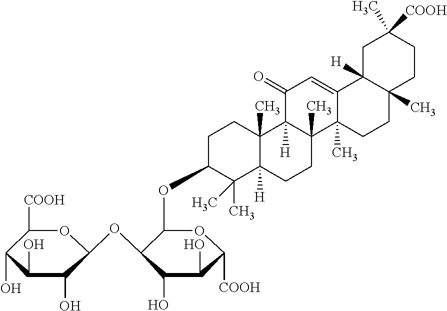

In certain embodiments, the term glycyrrhizin as used herein can refer to one of its pharmaceutically acceptable salts and/or racemic, enantiomeric, diastereoisomeric or tautomeric forms.

Examples of the pharmaceutically acceptable salts include but are not limited to ammonium salts such as monoammonium glycyrrhizinate, alkali metal salts such as disodium glycyrrhizinate, trisodium glycyrrhizinate and dipotassium glycyrrhizinate.

Glycyrrhizin can be found in some naturally-occuring extracts and/or plants, or chemically obtained (Shabkhiz M A, Eikani M R, Bashiri Sadr Z, Golmohammad F. Superheated water extraction of glycyrrhizic acid from licorice root. Food Chem. 2016, 210: 396-401).

In certain embodiments, glycyrrhizin can be extracted from a glycyrrhizin source such as a crude licorice. A glycyrrhizin source can be a glycyrrhizin-containing extract obtained from a plant material. In certain particular embodiments, a glycyrrhizin-containing extract can be obtained from a plant material containing glycyrrhizin, such as crude licorice, by an extraction method comprising at least the steps of:

(a) Providing a plant material containing glycyrrhizin;

(b) Grinding the plant material of step a), preferably in a solvent, thereby obtained a grinded mixture;

(c) Extracting glycyrrhizin from the grinded mixture, thereby obtaining a glycyrrhizin-containing extract;

(d) Optionally filtering the glycyrrhizin extract, thereby obtained a filtered glycyrrhizin extract;

(e) Optionally concentrating the filtered glycyrrhizin extract;

(f) Optionally spray-drying the filtered glycyrrhizin extract, thereby obtained a powder; and

(g) Optionally sieving the powder.

As used herein a “plant material containing glycyrrhizin” refers to a glycyrrhizin source. In certain particular embodiments, a plant material containing glycyrrhizin refers to a Glycyrrhiza glabra root. In certain more particular embodiments, a plant material containing glycyrrhizin refers to a glycyrrhizin-containing licorice extract or a glycyrrhizin-containing crude licorice.

As used herein a “solvent” can refer to acetic acid, n-butanol, isopropanol, n-propanol, ethanol, methanol, formic acid, dimethylformamide, dimethyl sulfoxide, acetonitrile, acetone, dichloromethane, tetrahydrofuran, ethyl acetate, n-hexane, benzene, toluene, diethyl ether, chloroform, 1,4-dioxane, water or a combination thereof. In certain particular embodiments, the solvent is water.

In certain embodiments, the term “plants” or “plant” encompasses Glycyrrhiza. Specifically, Glycyrrhiza is selected from the group consisting of Glycyrrhiza acanthocarpa, Glycyrrhiza aspera, Glycyrrhiza astragalina, Glycyrrhiza bucharica, Glycyrrhiza echinate, Glycyrrhiza eglandulosa, Glycyrrhiza eurycarpa, Glycyrrhiza foetida, Glycyrrhiza foetidissima, Glycyrrhiza frearitis, Glycyrrhiza glabra, Glycyrrhiza gontscharovii, Glycyrrhiza iconica, Glycyrrhiza inflata, Glycyrrhiza korshinskyi, Glycyrrhiza lepidota, Glycyrrhiza pallidiflora, Glycyrrhiza squamulose, Glycyrrhiza triphylla, Glycyrrhiza uralensis and Glycyrrhiza yunnanensis.

The term “licorice” or “liquorice”, as used herein, encompasses crude licorice, licorice root and Glycyrrhiza glabra root.

In certain embodiments, licorice can contain from about 1% to about 30% by weight of glycyrrhizin, relative to the total weight of dry matter of the licorice. In certain particular embodiments, licorice can contain from about 5% to about 20% by weight. In certain more particular embodiments, licorice can contain from about 6% to about 20% by weight of glycyrrhizin relative to the total weight of dry matter of the licorice.

According to certain embodiments, licorice root can contain from about 1% to about 25% by weight of glycyrrhizin, based on the total weight of dry matter of the licorice root.

According to certain particular embodiments, licorice root can contain from about 6% to about 12% of glycyrrhizin by weight, relative to the total weight of dry matter of the licorice root. As used herein, from about 6% to about 12% by weight of glycyrrhizin includes about 6.0%, about 6.5%, about 7.0%, about 7.5%, about 8.0%, about 8.5%, about 9.0%, about 9.5%, about 10.0%, about 10.5%, about 11.0%, about 11.5%, or about 12.0% by weight of glycyrrhizin, based on the total weight of dry matter of the licorice root.

In certain embodiments, licorice could be provided from a commercially available source such as the Licorice root PE 12% Glycyrrhizin preparation supplied by Naturex (Product ref; ED161596).

In a certain particular embodiment, the source of glycyrrhizin consists of a licorice, or extract thereof containing glycyrrhizin. Any other source of glycyrrhizin known by the person skilled in the art, especially any other source of glycyrrhizin apart from licorice, can also be used. In certain embodiments, the glycyrrhizin source can be pure glycyrrhizin.

In a general manner, the amount of glycyrrhizin in a given plant source, extract, or food composition, can be expressed in g/Mcal or in weight per total weight of dry matter of the given plant source, extract or composition.

In certain embodiments, the animal food composition or the kit includes a source of glycyrrhizin in an amount ranging from about 0.01 g/Mcal to about 10 g/Mcal. In certain particular embodiments, the animal food composition or the kit includes a source of glycyrrhizin in an amount of less than about 5 g/Mcal. In certain more particular embodiments, the animal food composition or the kit includes a source of glycyrrhizin in an amount of less than about 2 g/Mcal. In certain more particular embodiments, the animal food composition or the kit includes a source of glycyrrhizin in an amount of less than about 0.5 g/Mcal.

According to certain embodiments, the animal food composition or the kit comprises a source of glycyrrhizin in an amount ranging from about 0.01 g/Mcal to about 0.1 g/Mcal.

According to certain embodiments, the animal food composition or the kit includes a source of glycyrrhizin in an amount ranging from about 0.01% to about 10% by weight, relative to the total weight of dry matter of the animal food composition.

As used herein, from about 0.01% to about 4% by weight of a source of glycyrrhizin includes about 0.01%, about 0.1%, about 1%, about 2%, about 3% or about 4% by weight of a source of glycyrrhizin, based on the total weight of dry matter of the animal food composition.

According to certain embodiments, the animal food composition or the kit includes a source of glycyrrhizin in an amount ranging from about 0.01% to about 0.1% by weight based on the total weight of dry matter of the animal food composition.

As used herein, from about 0.01% to about 0.1% by weight of a source of glycyrrhizin includes about: 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.031%, 0.032%, 0.033%, 0.034%, 0.035%, 0.036%, 0.037%, 0.038%, 0.039%, 0.04%, 0.041%, 0.042%, 0.043%, 0.044%, 0.045%, 0.046%, 0.047%, 0.048%, 0.049%, 0.05%, 0.051%, 0.052%, 0.053%, 0.054%, 0.055%, 0.056%, 0.057%, 0.058%, 0.059%, 0.06%, 0.061%, 0.062%, 0.063%, 0.064%, 0.065%, 0.066%, 0.067%, 0.068%, 0.069%, 0.07%, 0.071%, 0.072%, 0.073%, 0.074%, 0.075%, 0.076%, 0.077%, 0.078%, 0.079%, 0.08%, 0.081%, 0.082%, 0.083%, 0.084%, 0.085%, 0.086%, 0.087%, 0.088%, 0.089%, 0.09%, 0.091%, 0.092%, 0.093%, 0.094%, 0.095%, 0.096%, 0.097%, 0.098%, 0.099%, or 0.1% by weight of a source of glycyrrhizin, based on the total weight of dry matter of the animal food composition.

### Curcuminoids

In certain embodiments, the food compositions and/or kits described herein can include one or more curcuminoids.

As used herein, the term “curcuminoid” refers to phenols that are present in the Indian spice turmeric. Turmeric is generally derived from the roots of the plant Curcuma longa. Curcuminoids have also been found in roots of other species in the plant family Zingiberaceae of the Curcuma genus. In particular, turmeric can contain from about 60% to about 80% in weight of curcumin, relative to the total weight of dry matter of turmeric, from about 15% to about 30% in weight of demethoxycurcumin, relative to the total weight of dry matter of turmeric, and from about 2% to about 6% in weight of bis-demethoxycurcumin, relative to the total weight of dry matter of turmeric. The curcuminoid in the food composition or the kit of the present disclosure can be of any format, including a powder or lipid extract.

In certain embodiments, curcuminoids can be found in some naturally-occuring extracts and/or plants, or chemically obtained.

In a certain particular embodiment, the source of curcuminoids consists of a turmeric extract, i.e., Curcuma Longa. Non-limitative examples of turmeric extract are the turmeric extract BCM-95® from Arjuna or the turmeric extract from Naturex. Any other source of curcuminoids known to a person skilled in the art can also be used.

Other available sources for curcuminoids include but are not limited to liposomal curcumin, curcumin nanoparticles, curcumin phospholipid complex (e.g., MERIVA sourcing, about 20% total curcuminoids to BCM95 Arjuna with about 90% in weight of total curcuminoids), structural analogues of curcumin (e.g., EF-24) demethoxycurcumin, bisdemethoxycurcumin, tetrahydrocurcumin, and commercial/DM, and any formulation designed to enhance curcumin bioavailability.

In certain embodiments, curcuminoids can be found in other botanicals in addition to Curcuma longa, such as Curcuma xanthorrhiza and Curcuma zedoania. Curcuminoid in its pure form has poor solubility in water. Curcuminoids can be extracted from curcuminoids-containing plants, e.g., turmeric root, with organic solvent such as ethanol or acetone.

In particular embodiments, curcuminoids according to the present disclosure can include compounds of the general formula (I), pharmaceutically acceptable salts, tautomers and stereoisomers thereof:

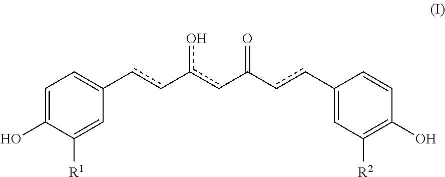

wherein R¹ and R² are independently chosen from hydrogen atom, methoxy, methyl, hydroxyl and ethoxy group.

In certain embodiments, R¹ and R² are the same. In certain other embodiments, R¹ and R² are different.

In certain particular embodiments, the curcuminoids are selected from the group consisting of curcumin (II), demethoxycurcumin (III), bis-methoxycucurmin (IV), tetrahydrocurcumin (V) and combinations thereof. Structures of each of the compounds (II)-(V) are provided below:

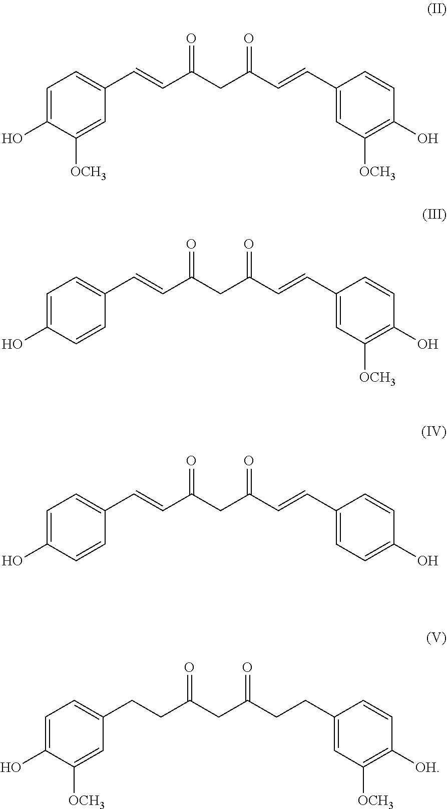 [SMILES: COc1cc(/C=C/C(=O)CC(=O)/C=C/c2ccc(O)c(OC)c2)ccc1O ; COc1cc(/C=C/C(=O)CC(=O)/C=C/c2ccc(O)cc2)ccc1O ; O=C(/C=C/c1ccc(O)cc1)CC(=O)/C=C/c1ccc(O)cc1 ; COc1cc(CCC(=O)CC(=O)CCc2ccc(O)c(OC)c2)ccc1O]

In a certain particular embodiment, curcuminoid is curcumin.

In a certain particular embodiment, the source of curcuminoids (e.g., curcumin) is turmeric.

According to certain embodiments, the animal food composition or the kit comprises a source of curcuminoids in an amount ranging from about 0.01 g/Mcal to about 10 g/Mcal. In particular embodiments, the animal food composition or the kit comprises a source of curcuminoids in an amount of less than about 5 g/Mcal. In certain particular embodiments, the animal food composition or the kit comprises a source of curcuminoids in an amount of less than about 2 g/Mcal. In certain particular embodiments, the animal food composition or the kit comprises a source of curcuminoids in an amount of less than about 0.5 g/Mcal. In certain embodiments, the animal food composition or the kit comprises a source of curcuminoids in an amount ranging from about 0.01 g/Mcal to about 0.1 g/Mcal

In certain embodiments, the animal food composition or the kit comprises a source of curcuminoids in an amount ranging from about 0.01% to about 10% by weight, based on the total weight of dry matter of the food composition.

As used herein, from about 0.01% to about 10% by weight of a source of curcuminoids includes about 0.01%, about 0.1%, about 1%, about 2%, about 3%, about 4%, about 5%, about 6%, about 7%, about 8%, about 9%, or about 10% by weight of a source of curcuminoids, based on the total weight of dry matter of the food composition.

In certain embodiments, the curcuminoid source can consist of pure curcuminoids.

In certain embodiments, the animal food composition or the kit includes a source of curcuminoids in an amount ranging from about 0.01% to about 0.1% by weight, relative to the total weight of dry matter of the animal food composition.

As used herein, from about 0.01% to about 0.1% of a source of curcuminoids includes about: 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.031%, 0.032%, 0.033%, 0.034%, 0.035%, 0.036%, 0.037%, 0.038%, 0.039%, 0.04%, 0.041%, 0.042%, 0.043%, 0.044%, 0.045%, 0.046%, 0.047%, 0.048%, 0.049%, 0.05%, 0.051%, 0.052%, 0.053%, 0.054%, 0.055%, 0.056%, 0.057%, 0.058%, 0.059%, 0.06%, 0.061%, 0.062%, 0.063%, 0.064%, 0.065%, 0.066%, 0.067%, 0.068%, 0.069%, 0.07%, 0.071%, 0.072%, 0.073%, 0.074%, 0.075%, 0.076%, 0.077%, 0.078%, 0.079%, 0.08%, 0.081%, 0.082%, 0.083%, 0.084%, 0.085%, 0.086%, 0.087%, 0.088%, 0.089%, 0.09%, 0.091%, 0.092%, 0.093%, 0.094%, 0.095%, 0.096%, 0.097%, 0.098%, 0.099%, or 0.1% of a source of curcuminoids by weight, based on the total weight of dry matter of the animal food composition.

### Further Ingredients

According to the first aspect of the present disclosure, the animal food composition or the kit for preventing and/or treating allergic inflammatory skin diseases further includes linoleic acid.

In certain embodiments, the animal food composition or the kit further comprises EPA/DHA, Taurine, Lutein, Vitamin E and/or a combination thereof.

In certain embodiments, the animal food composition or the kit can additionally further include one or more vitamin A, vitamin B3, vitamin C, zinc, vitamin D, one or more fatty acids and/or a combination thereof.

### Linoleic Acid

In certain embodiments, the animal food composition and/or kit described herein can include linoleic acid. As used herein, the term “linoleic acid” refers to a polyunsaturated omega-6 fatty acid, (9Z,12Z)-octadeca-9,12-dienoic acid, which is one of two essential fatty acids for animals.

For measuring the content of a food composition in linoleic acid, the one skilled in the art can refer to any of well-known techniques. For example, a method based upon the norm NF EN ISO 5508/5509 via chromatography in gaseous phase can be used.

In certain embodiments, the animal food composition or the kit described herein comprises one or more substances, which are sources of linoleic acid.

Linoleic acid sources, i.e., linoleic acid-containing substances, suitable for the presently disclosed application include but are not limited to vegetable oils, although animal oils or crude fats can also be used. In particular embodiments, linoleic sources, i.e., linoleic acid-containing substances, include safflower oil, sunflower oil, soybean oil, sesame oil, canola oil, other plant or animal oils/crude fats, meats or a combination of two or more thereof.

In certain particular embodiments, linoleic acid sources, i.e., linoleic acid-containing substances, that are included in the animal food composition or the kit described herein, are selected from the group consisting of: safflower oil, sunflower oil, soybean oil, sesame oil, canola oil, meats or a combination thereof.

In certain embodiments, the animal food composition or the kit described herein includes linoleic acid in an amount ranging from about 1 g/Mcal to about 20 g/Mcal or from about 2% to about 10% by weight, relative to the total weight of dry matter of the animal food composition.

In certain embodiments, linoleic acid is present in the animal food composition or the kit in an amount of from about 2 g/Mcal to about 20 g/Mcal, from about 3 g/Mcal to about 20 g/Mcal, from about 4 g/Mcal to about 20 g/Mcal, from about 5 g/Mcal to about 20 g/Mcal, from about 6 g/Mcal to about 20 g/Mcal, from about 7 g/Mcal to about 20 g/Mcal, from about 8 g/Mcal to about 20 g/Mcal, from about 9 g/Mcal to about 20 g/Mcal, from about 10 g/Mcal to about 20 g/Mcal, from about 11 g/Mcal to about 20 g/Mcal, from about 12 g/Mcal to about 20 g/Mcal, from about 13 g/Mcal to about 20 g/Mcal, from about 14 g/Mcal to about 20 g/Mcal, from about 15 g/Mcal to about 20 g/Mcal, from about 16 g/Mcal to about 20 g/Mcal, from about 17 g/Mcal to about 20 g/Mcal, from about 18 g/Mcal to about 20 g/Mcal, from about 19 g/Mcal to about 20 g/Mcal, from about 1 g/Mcal to about 19 g/Mcal, from about 1 g/Mcal to about 18 g/Mcal, from about 1 g/Mcal to about 17 g/Mcal, from about 1 g/Mcal to about 16 g/Mcal, from about 1 g/Mcal to about 15 g/Mcal, from about 1 g/Mcal to about 14 g/Mcal, from about 1 g/Mcal to about 13 g/Mcal, from about 1 g/Mcal to about 12 g/Mcal, from about 1 g/Mcal to about 11 g/Mcal, from about 1 g/Mcal to about 10 g/Mcal, from about 1 g/Mcal to about 9 g/Mcal, from about 1 g/Mcal to about 8 g/Mcal, from about 1 g/Mcal to about 7 g/Mcal, from about 1 g/Mcal to about 6 g/Mcal, from about 1 g/Mcal to about 5 g/Mcal, from about 1 g/Mcal to about 4 g/Mcal, from about 1 g/Mcal to about 3 g/Mcal, from about 1 g/Mcal to about 2 g/Mcal, from about 2 g/Mcal to about 19 g/Mcal, from about 5 g/Mcal to about 15 g/Mcal, from about 7.5 g/Mcal to about 12.5 g/Mcal, or about 10 g/Mcal.

In certain embodiments, linoleic acid is present in the animal food composition or the kit in an amount of from about 2% to about 10%, from about 3% to about 10%, from about 4% to about 10%, from about 5% to about 10%, from about 6% to about 10%, from about 7% to about 10%, from about 8% to about 10%, from about 9% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 2% to about 9%, from about 3% to about 8%, from about 4% to about 7%, or about 5% by weight, relative to the total weight of dry matter of the animal food composition.

### EPA/DHA

In certain embodiments, the animal food composition and/or kit described herein can include EPA/DHA. As used herein, the term “EPA/DHA” designates a fatty acid or a mixture of fatty acids consisting of (i) only eicosapentaenoic acid (EPA), (ii) only docosahexaenoic acid (DHA) or (iii) a combination of eicosapentaenoic acid and docosahexaenoic acid (EPA+DHA).

Accordingly, an amount of “EPA/DHA” refers to EPA, or DHA or a combination of EPA and DHA.

In certain embodiments, the animal food composition or the kit as described herein, includes a combination of Eicosapentaenoic acid (EPA) and Docosahexaenoic acid (DHA) in a weight ratio of EPA:DHA of from about 0.1 to about 100.

According to further embodiments, there is no specific requirement regarding the respective amount of EPA and DHA present in the EPA/DHA combination. Illustratively, the weight/energy ratio of EPA to DHA can range from 0.0001 to 1000, relative to the total weight of dry matter of the animal food composition.

In certain embodiments, the animal food composition or the kit disclosed herein includes EPA/DHA in an amount ranging from about 0.1 g/Mcal to about 7 g/Mcal or from about 0.1% to about 5% by weight, relative to the total weight of dry matter of the animal food composition.

In certain embodiments, EPA/DHA is present in the animal food composition or the kit in an amount of from about 0.1 g/Mcal to about 7 g/Mcal, from about 0.5 g/Mcal to about 7 g/Mcal, from about 1 g/Mcal to about 7 g/Mcal, from about 1.5 g/Mcal to about 7 g/Mcal, from about 2 g/Mcal to about 7 g/Mcal, from about 2.5 g/Mcal to about 7 g/Mcal, from about 3 g/Mcal to about 7 g/Mcal, from about 3.5 g/Mcal to about 7 g/Mcal, from about 4 g/Mcal to about 7 g/Mcal, from about 4.5 g/Mcal to about 7 g/Mcal, from about 5 g/Mcal to about 7 g/Mcal, from about 5.5 g/Mcal to about 7 g/Mcal, from about 6 g/Mcal to about 7 g/Mcal, from about 6.5 g/Mcal to about 7 g/Mcal, from about 0.1 g/Mcal to about 6.5 g/Mcal, from about 0.1 g/Mcal to about 6 g/Mcal, from about 0.1 g/Mcal to about 5.5 g/Mcal, from about 0.1 g/Mcal to about 5 g/Mcal, from about 0.1 g/Mcal to about 4.5 g/Mcal, from about 0.1 g/Mcal to about 4 g/Mcal, from about 0.1 g/Mcal to about 3.5 g/Mcal, from about 0.1 g/Mcal to about 3 g/Mcal, from about 0.1 g/Mcal to about 2.5 g/Mcal, from about 0.1 g/Mcal to about 2 g/Mcal, from about 0.1 g/Mcal to about 1.5 g/Mcal, from about 0.1 g/Mcal to about 1 g/Mcal, from about 0.1 g/Mcal to about 0.5 g/Mcal, from about 0.5 g/Mcal to about 6.5 g/Mcal, from about 1 g/Mcal to about 6 g/Mcal, from about 1.5 g/Mcal to about 5.5 g/Mcal, from about 2 g/Mcal to about 5 g/Mcal, from about 2.5 g/Mcal to about 4.5 g/Mcal, from about 2.5 g/Mcal to about 4.5 g/Mcal, from about 3 g/Mcal to about 4 g/Mcal, or about 3.5 g/Mcal.

In certain embodiments, EPA/DHA is present in the animal food composition or the kit in an amount of from about 0.1% to about 5%, from about 0.5% to about 5%, from about 1% to about 5%, from about 1.5% to about 5%, from about 2%, to about 5%, from about 2.5% to about 5%, from about 3% to about 5%, from about 3.5% to about 5%, from about 4% to about 5%, from about 4.5% to about 5%, from about 0.1% to about 4.5%, from about 0.1% to about 4%, from about 0.1% to about 3.5%, from about 0.1% to about 3%, from about 0.1% to about 2.5%, from about 0.1% to about 2%, from about 0.1% to about 1.5%, from about 0.1% to about 1%, from about 0.1% to about 0.5%, from about 0.5% to about 4.5%, from about 1% to about 4% or from about 1.5% to about 3.5% by weight, relative to the total weight of dry matter of the animal food composition.

### Taurine

In certain embodiments, the animal food composition and/or kit described herein can include taurine. Taurine is a non-essential amino acid which is obtained from meat and fish. It stimulates the production of glycosphingolipids in the skin by acting as a precursor molecule. Glycosphingolipids exhibit anti-microbial properties.

In certain embodiments, taurine according to the present disclosure refers to a compound of the following structure

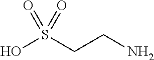

or one of its pharmaceutically acceptable salts and/or racemic, enantiomeric, diastereoisomeric or tautomeric forms.

### Lutein

In certain embodiments, the animal food composition and/or kit described herein can include lutein. As used herein, “lutein” refers to a xanthophyll and one of known naturally occurring carotenoids. Lutein is a lipophilic molecule and is generally insoluble in water. Lutein is synthesized only by plants and like other xanthophylls is found in high quantities in green leafy vegetables such as spinach, kale and yellow carrots. Lutein is isomeric with zeaxanthin, differing only in the placement of one double bond. The principal natural stereoisomer of lutein is (3R,3′R,6′R)-beta, epsilon-carotene-3,3′-diol. Lutein is present in plants as fatty-acid esters, with one or two fatty acids bound to the two hydroxyl-groups.

In certain embodiments, lutein according to the present disclosure refers to a compound of the following structure:

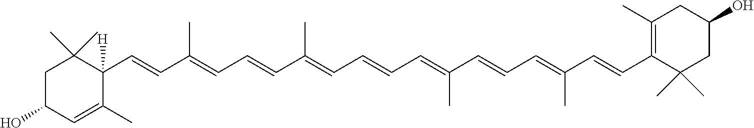 [SMILES: CC1=C[C@H](O)CC(C)(C)[C@H]1/C=C/C(C)=C/C=C/C(C)=C/C=C/C=C(C)/C=C/C=C(C)/C=C/C1=C(C)C[C@@H](O)CC1(C)C]

or one of its pharmaceutically acceptable salts and/or racemic, enantiomeric, diastereoisomeric or tautomeric forms.

### Vitamin E

In certain embodiments, the animal food composition and/or kit described herein can include vitamin E. Vitamin E is a collective term for several biologically similar compounds, including those called tocopherols and tocotrienols, which share the same biological activity. The most biologically active biological form of Vitamin E (also the most active antioxidant) in animal tissue is alpha-tocopherol. Vitamin E cannot be synthesized in vivo. Vitamin E protects against the loss of cell membrane integrity, which adversely alters cellular and organelle function. Vitamin E according to an aspect of the present disclosure can be in any form. It can be liquid, semi-solid or solid. It can be a tocopherol or a tocotrienol. It can be alphatocopherol, (d-x or dl-oc) beta-tocopherol (d-,B or dl-), gamma-tocopherol (dot or dim), delta-tocopherol, alpha-tocotrienol, beta-tocotrienol, gamma-tocotrienol or delta-tocotrienol. In certain particular embodiments, Vitamin E is alpha-tocopherol.

According to one embodiment, the source of the Vitamin E is not limiting. In certain embodiments, Vitamin E sources include Vitamin E acetate, (e.g., tocopherol acetate), Vitamin E acetate adsorbate or Vitamin E acetate spray dried. In certain particular embodiments, sources of Vitamin E are synthetic, although natural sources can be used.

In certain embodiments, Vitamin E according to the present disclosure refers to a compound of the following structure:

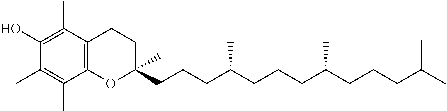 [SMILES: Cc1c(C)c2c(c(C)c1O)CC[C@@](C)(CCC[C@H](C)CCC[C@H](C)CCCC(C)C)O2]

or one of its pharmaceutically acceptable salts and/or racemic, enantiomeric, diastereoisomeric or tautomeric forms.

### Supplementary Ingredients, Substances or Additives

In some embodiments, the animal food composition or the kit as described herein is nutritionally complete and can also contain supplementary ingredients, substances or additives, for example, protein, crude fat, crude fibers, NFE, ash, minerals, vitamins or condiments.

### Protein

In certain embodiments, an animal food composition or the kit according to the present disclosure can further contain a protein source. In certain embodiments, the protein level is high enough so as to ensure maintenance of lean body mass of the animal. In certain embodiments, the animal food composition or the kit of the present disclosure can contain one or more distinct protein sources.

In certain embodiments, an animal food composition as described herein can comprise a plurality of proteins that are contained in a protein source which is used in the manufacture process.

In certain other embodiments, the animal food composition as described herein can comprise a plurality of proteins from a plurality of protein sources.

In certain embodiments the protein is not hydrolyzed. In certain other embodiments a protein can be present in an at least partially hydrolysed form, or even completely hydrolyzed. In certain embodiments, the animal food composition or the kit according to the present disclosure can incorporate proteins under the form of meat or animal derived material (such as, but not limited to beef, chicken, turkey, lamb, fish, blood plasma, marrow bone etc. or combinations thereof). In certain other embodiments, the animal food composition or the kit as described herein can be meat-free. In certain embodiments, such meat-free compositions/kits include a meat substitute protein source such as soya, maize gluten or any other protein-containing soya product in order to provide a protein source. In certain embodiments, the animal food composition or the kit as disclosed herein can include additional protein sources such as soya protein concentrate, milk proteins, gluten etc.

### Crude Fat

An animal food composition or a kit according to the present disclosure can further contain a nutritionally appropriate amount of crude fat.

The expression “crude fat” as used in the present specification refers to any food-acceptable crude fat(s) and/or oil (s) irrespective of their consistency at room temperature, i.e., irrespective whether said “crude fat” is present in essentially fluid form or in essentially solid form. The animal food composition or the kit according to the present disclosure can include crude fat of animal and/or vegetable origin. Crude fat can be supplied by any of a variety of sources known by those skilled in the art. Plant crude fat sources include, without limitation, wheat, sunflower, safflower, rapeseed, olive, borage, flaxseed, peanuts, blackcurrant seed, cottonseed, wheat, germ, corn germ as well as oils derived from these and other plant crude fat sources. Animal sources include, for example and without limitation, chicken crude fat, turkey crude fat, beef crude fat, duck crude fat, pork crude fat, lamb crude fat, etc., fish oil or any meat, meat by-products, seafood, dairy, eggs, etc. Crude fat content of foods can be determined by any number of methods known by those of skill in the art.

### Fibers

Fibers are optionally comprised in an animal food composition or a kit disclosed herein. The expression “fibers” is used herein similarly to “dietary fibers” and shall be interpreted for the purpose of the present disclosure as Total Fibers, meaning that it includes soluble fibers and insoluble fibers. Soluble fiber, as used herein, refers to a fiber that is resistant to digestion and absorption in the small intestine and undergo complete or partial fermentation in the large intestine. In contrast, insoluble fiber. As used herein refers to non-starch polysaccharides that are resistant to digestion and absorption in the small intestine, and resistant to fermentation in the large intestine. Soluble fibers are considered as having a prebiotic effect by providing short chain fatty acids as a source of energy to colonocytes. Insoluble fibers are considered as useful for transit and ballast effect. As non-limiting examples of fibers include a first group, which includes beet pulp, guar gum, chicory root, psyllium, pectin, blueberry, cranberry, squash, apples, oats, beans, citrus, barley, or peas, and a second group, which includes cellulose, whole wheat products, wheat oat, corn bran, flax seed, grapes, celery, green beans, cauliflower, potato skins, fruit skins, vegetable skins, peanut hulls, and soy fiber.

### Nitrogen Free Extract (NFE)

In certain embodiments, the animal food composition and/or kit described herein can include nitrogen free extract (NFE). As used herein, and as conventionally admitted in the art, the Nitrogen Free Extract (NFE) refers to the soluble carbohydrate fraction that can be optionally included in an animal food composition or a kit disclosed herein. NFE can encompasses a wide variety of soluble polysaccharides, starch, gums, mucilages and pectin, if present in the animal food composition or the kit disclosed herein. As conventionally known in the art, NFE does not comprise the insoluble carbohydrate fraction comprised in the crude fiber material that can, in some embodiments, be present in the food compositions or kits of the present disclosure.

Typically, the content of a food composition in Nitrogen Free Extract is determined by subtracting the content of each of the other components (protein, crude fat, crude fiber, ash) from the whole dry matter of the said food composition.

In certain embodiments where the qualitative and quantitative features of a food composition is expressed as the energy density (e.g., the metabolizable energy density in g/Mcal), the NFE content is determined by subtracting the energy value of each of the other components (protein, crude fat, crude fiber, ash) from the energy value of the whole food composition.

In certain embodiments wherein the qualitative and quantitative features of a food composition is expressed as a weight percentage (e.g., a weight percentage based on the total weight of dry matter of the said composition), the NFE content is determined by subtracting the weight percentage of the other components (protein, crude fat, crude fiber, ash) from the total weight of the said food composition.

In certain embodiments, where the qualitative and quantitative features of a food composition are expressed as a weight percentage (e.g., a weight percentage based on the total weight of dry matter of the said composition), the NFE content is determined by subtracting the weight percentage of the other components (protein, fat, crude fiber, ash) from the total weight of the said food composition.

### Carbohydrates

In certain embodiments, the animal food composition and/or kit described herein can include carbohydrates. As used herein, the term “carbohydrates” designates a mixture of polysaccharides and sugars that are metabolized for energy when hydrolyzed in the body. The carbohydrate content in the food composition and/or the kit can be determined by any number of methods known by those of skilled in the art. Carbohydrates can be supplied under the form of any of a variety of carbohydrate sources known by those skilled in the art, including starch (any kinds, corn, wheat, barley, etc.) beet pulp (which contain a bit of sugars) and psyllium.

### Starches

An important source of Nitrogen Free Extract that is optionally comprised in an animal food composition and/or kit disclosed herein consists of starch. A person skilled in the art will appreciate a wide variety of starches are suitable for use in compositions of the present disclosure.

The term “starch” as used herein refers to a polysaccharide that is composed of amylose and amylopectin. Starch occurs in many plant tissues as granules, usually between 1 and 100 μm in diameter, depending upon the plant source. Chemically, starches are polysaccharides composed of α-D-glucopyranosyl units linked together with α-D(1-4) and/or α-D(1-6) linkages and are comprised of two molecular types: amylose, the straight chain polyglucan comprised of approximately 1000, α-D(1-4) linked glucoses; and amylopectin, the branched glucan, comprised of approximately 4000 glucose unite with branches occurring as α-D(1-6) linkages.

As used herein, starch encompasses the various crystalline structures of A-type, B-type and C-type starches, which contain different proportions of amylopectin. A-type starches are found mainly in cereals, while B-type starches are found mainly in tubers and amylose-rich starches. C-type starch consists of a mixture of both A and B forms and is found mainly in legumes.

In general, digestible starches are broken down (hydrolyzed) by the enzymes a-amylases, glucoamylase and sucrose-iso-maltase in the small intestine to yield free glucose that is then absorbed.

Starch comprised in an animal food or kits disclosed herein can consist in any starch suitable for dietary purpose.

Indeed, the native starch comprised in the starting materials used for preparing an animal food composition or a kit as described herein is susceptible to undergo changes during the manufacture process. The specified amount of starch comprised in an animal food composition or a kit described in the present disclosure consists of the amount of starch that is contained in the total amount of the raw materials which are provided for producing the said animal food composition or kit. However, the amount of starch comprised in an animal food composition or the kit disclosed herein equates the total amount of starch comprised in the starting materials used for preparing the said animal food composition or kit.

If not already known by advance, the starch content of the starting materials used for preparing an animal food composition or a kit described herein can be determined according to conventional techniques known in the art, and especially according to the known polarimetric method, such as according to NF EN ISO 10520.

In certain embodiments, wherein the starting materials can comprise also modified starch or pre-gelatinized starch, the starch content can also be determined according to NF EN ISO 15914.

### Ash

In certain embodiments, the food compositions and/or the kits disclosed herein can include ash. Ash can include minerals, such as calcium, phosphorus, sodium, chloride, potassium, magnesium, or combinations thereof. The ash content, if specified as a result of an analytical measure of an animal food composition or a kit described herein, is a measure of the total amount of minerals comprised therein. The mineral content is a measure of the amount of specific inorganic components comprised therein, which includes calcium (Ca), sodium (Na), potassium (K) and chlorine (Cl).

As it shall be readily understood, every embodiment of an animal food composition or a kit encompassed by the present disclosure comprises a variety of ingredients, each comprised in the said composition or kit at a given weight percentage, as compared to the total weight of dry matter of the animal food composition.

In certain embodiments, an animal food composition or a kit as disclosed herein comprises, protein, crude fat, one more sources of EPA/DHA, ash, fibre, NFE and optionally one or more further ingredients such as vitamins, minerals, etc., the sum of the weight of each of the ingredients comprised therein amounting to 100% by weight, based on the total weight of dry matter of the said animal food composition or kit.

### Animal Food Compositions and Kits

The present disclosure further relates to an animal food composition or a kit as described herein, as such, comprising:

(i) a source of glycyrrhizin, and

(ii) a source of one or more curcuminoids.

According to one particular embodiment, when the composition is in the form of a kit, this kit can comprise:

(i) a first part comprising a source of glycyrrhizin; and

(ii) a second part comprising a source of one or more curcuminoids.

The animal food compositions and kits described herein can be as described above.

In a particular embodiment, the animal food composition includes:

(i) a source of glycyrrhizin, in combination with (ii) a source of one or more curcuminoids; wherein the animal food composition consists of a kibble and/or the animal food composition further comprising a source of proteins.

As detailed elsewhere, in certain embodiments, a source of glycyrrhizin can consist of at least one of the plants of genus Glycyrrhiza. In particular embodiments, a source of glycyrrhizin is Glycyrrhiza glabra root, e.g., licorice. In certain particular embodiments, a source of glycyrrhizin is licorice.

In certain embodiments, and as also detailed elsewhere, a source of glycyrrhizin is present in the animal food composition in an amount ranging from about 0.01 g/Mcal to about 0.1 g/Mcal, based on the total weight of dry matter of the animal food composition.

In certain embodiments, and as also detailed elsewhere, a source of glycyrrhizin is present in the animal food composition in an amount ranging from about 0.01% to about 10% by weight, based on the total weight of dry matter of the animal food composition.

In certain embodiments, a curcuminoid is a compound represented by the general formula (I), a pharmaceutically acceptable salt, a stereoisomer or a tautomer thereof:

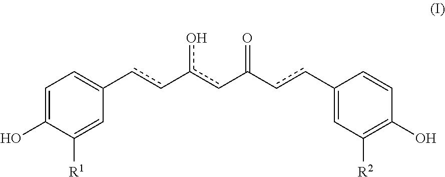

wherein R¹ and R² are independently chosen from hydrogen atom, methoxy, methyl, hydroxyl and ethoxy group.

In certain embodiments, the one or more curcuminoids are selected from the group consisting of curcumin, demethoxycurcumin, bis-methoxycurcumin and/or tetrahydrocurcumin or combinations thereof.

In certain particular embodiments, the one or more curcuminoids consist of curcumin.

In certain embodiments, Curcuma longa, Curcuma xanthorrhiza and Curcuma zedoania are some of the sources of curcuminoids. In particular embodiments, the source of the one or more curcuminoids is turmeric.

Illustratively, in certain embodiments, and as also detailed elsewhere, a source of curcuminoid can be in an amount ranging from about 0.01 g/Mcal to about 0.1 g/Mcal, relative to the total weight of dry matter of the composition.

Illustratively, in certain embodiments, and as also detailed elsewhere, a source of curcuminoid can be in an amount ranging from about 0.01% to about 10% by weight, relative to the total weight of dry matter of the composition.

In certain embodiments, an animal food composition or a kit as described herein consists of a kibble. In certain embodiments, an animal food composition or a kit as described herein further includes a source of proteins.

In certain particular embodiments, the disclosure relates to an animal food composition comprising:

(i) a source of glycyrrhizin, in combination with

(ii) a source of one or more curcuminoids;

wherein the animal food composition is a kibble and/or the animal food composition further comprising a source of proteins.

In a particular embodiment the animal food composition or kit consists of a nutritionally complete animal food composition.

In a particular embodiment the animal food composition or kit consists of a food supplement and/or a drug.

In a particular embodiment, the source of glycyrrhizin consists of licorice, and/or the source of one or more curcuminoids consists of turmeric.

In certain embodiments, the animal food composition or kit as described herein further includes linoleic acid. In a particular embodiment, the source of linoleic acid is selected from the group consisting of safflower oil, sunflower oil, soybean oil, sesame oil, canola oil, meats or a combination thereof.

In certain other embodiments, the animal food composition or kit further includes EPA/DHA, taurine, lutein, Vitamin E and/or a combination thereof.

In certain other embodiments, the animal food composition or kit include or consist of dry, wet and/or semi-moist food compositions.

In certain other embodiments, the animal food composition or kit includes:
licorice in an amount ranging from about 0.02 g/Mcal to about 1 g/Mcal. from about 0.04 g/Mcal to about 0.07 g/Mcal or about 0.054 g/Mcal, and
turmeric in an amount from about 0.05 g/Mcal to about 0.08 g/Mcal, or about 0.68 g/Mcal.

In certain embodiments, the animal food composition or kit of the present disclosure includes:
licorice in an amount ranging from about 0.02 g/Mcal to about 0.1 g/Mcal,
turmeric in an amount ranging from about 0.05 g/Mcal to about 0.08 g/Mcal,
linoleic acid in an amount ranging from about 8 g/Mcal to about 12 g/Mcal,
EPA/DHA in an amount ranging from about 1 g/Mcal to about 2 g/Mcal,
Taurine in an amount ranging from about 1 g/Mcal to about 2 g/Mcal,
Lutein in an amount ranging from about 0.001 g/Mcal to about 0.002 g/Mcal, and/or
Vitamin E in an amount from about 0.2 g/Mcal to about 0.3 g/Mcal.

Illustratively, in certain embodiments, the animal food composition includes:
licorice in an amount of about 0.054 g/Mcal,
turmeric in an amount of about 0.068 g/Mcal,
linoleic acid in an amount of about 10.5 g/Mcal,
EPA/DHA in an amount of about 1.5 g/Mcal,
Taurine in an amount of about 1.2 g/Mcal,
Lutein in an amount of about 0.0013 g/Mcal, and/or
Vitamin E in an amount of about 0.244 g/Mcal.

In certain embodiments, the animal is a canine. In certain particular embodiments, the animal is a dog.

### Methods for the Preparation of a Composition

The present disclosure also provides a method for manufacturing the animal food composition specified herein. The process for the manufacture of the animal food composition as described includes any methods known in the art.

The animal food composition described herein can be manufactured by mixing together ingredients and kneading in order to make consistent dough or meat emulsion that can be cooked. This applies also to liquids where ingredients are mixed, homogenized before a cooking step in a packaging. The process of creating an embodiment of a dry food is usually done by baking and/or extruding. The dough is typically fed into a machine called an expander and/or extruder, which uses pressurized steam or water to cook the ingredients. While inside the extruder, the dough is under extreme pressure and high temperatures. The dough is then pushed through a die (specifically sized and shaped hole) and then cut off using a knife. The puffed dough pieces are made into kibble by passing it through a dryer so that moisture is dropped down to a defined target ensuring stability of the food until consumption. The kibble can then be sprayed with fats, oils, minerals, vitamins, the natural extracts cocktail, palatants and optionally sealed into packages.

The composition can be a dry food, a wet food or a semi-moist food as described in the present disclosure.

In certain embodiments, the present disclosure provides a method of manufacturing an animal food composition comprising (i) a source of glycyrrhizin, in combination with (ii) a source of one or more curcuminoids; wherein it includes the steps of:

a) mixing a source of glycyrrhizin and a source of one or more curcuminoids, thereby providing a mixture; and

b) heating the mixture.

In certain embodiments, the animal food composition manufactured by the methods described herein, is a kibble. In certain embodiments, the animal food composition manufactured by the methods described herein includes a source of proteins.

In a particular embodiment, the source of glycyrrhizin and the source of one or more curcuminoids are mixed in step a), with ingredients selected from the group consisting of a source of linoleic acid, EPA/DHA, taurine, lutein, Vitamin E and a combination thereof.

In a certain particular embodiment, the source of glycyrrhizin and the source of curcuminoids are mixed with ingredients selected from the group consisting of a source of linoleic acid, EPA/DHA, taurine, lutein, Vitamin E, protein, crude fat, fibers and a combination thereof.

### Therapeutic or Non-Therapeutic Applications

In further embodiments, the present disclosure provides the use of an animal food composition or a kit as described herein for preventing and/or treating allergic inflammatory skin diseases. In particular embodiments, the present disclosure provides the use of an animal food composition or a kit as described herein for preventing and/or treating atopic dermatitis, including atopic dermatitis in canine animals, and particularly, in dogs.

In certain embodiments, the present disclosure relates to an animal food composition or kit comprising:

(i) a source of glycyrrhizin, in combination with

(ii) a source of one or more curcuminoids;

for use in a method for preventing and/or treating allergic inflammatory skin diseases.

In certain other embodiments, an animal food composition or a kit of the present disclosure can be used in a therapeutic or a non-therapeutic method for preventing and/or treating allergic inflammatory skin diseases.

As previously mentioned herein, allergic inflammatory skin diseases can be selected from the group consisting of atopic dermatitis, flea allergic dermatitis, urticaria, angioedema, inhalant allergy, inhalant allergic dermatitis, food allergic dermatitis, contact dermatitis, miliary dermatitis, eosinophilic granuloma, head and neck pruritus and generalized pruritus. In certain particular embodiments, allergic inflammatory skin diseases are atopic dermatitis, including atopic dermatitis in canine animals, especially in dogs.

As detailed elsewhere in the present specification and as shown in the Examples, the animal food composition or kit described herein acts positively on the various three (3) outcomes associated with allergic inflammatory skin disease, especially atopic dermatitis, more especially in canine atopic dermatitis.

In certain other embodiments, the animal food composition or kit described herein is used in a method for preventing and/or treating an animal affected with an allergic inflammatory skin disease. In particular embodiments, the animal is affected with an atopic dermatitis, e.g., a canine atopic dermatitis.

In a certain particular embodiment, the animal food composition is used for preventing and/or treating atopic dermatitis.

In certain other embodiments, the animal food composition is used for preventing and/or treating canine atopic dermatitis.

In certain other embodiments, the animal food composition is used for decreasing pruritus flares.

In certain other embodiments, the animal food composition is used for decreasing the CADESI-04 score.

In certain other embodiments, the animal food composition is used for reducing the dose and frequency of drugs required to manage the condition of an allergic inflammatory skin disease.

The present disclosure also includes a kit comprising a source of glycyrrhizin and a source of one or more curcuminoids for use in a method for preventing and/or treating allergic inflammatory skin diseases. In particular embodiments, the allergic inflammatory skin disease is atopic dermatitis, e.g., canine atopic dermatitis.

The present disclosure also provides for a therapeutic or a non-therapeutic method for preventing and/or treating allergic inflammatory skin diseases in an animal including at least a step of feeding the animal with an animal food composition or a kit according to the present disclosure.

In a certain embodiment, the animal is affected with an allergic inflammatory skin disease. In a particular embodiment, the animal is affected with an atopic dermatitis. In a certain particular embodiment, the animal is a dog affected with a canine atopic dermatitis.

As shown in the Examples, an animal food composition or kit as described herein allows preventing or treating allergic inflammatory skin diseases, such as atopic dermatitis. Beneficial effects are obtained (i) in animals fed exclusively with the food composition or kit described herein, (ii) in animals fed partly with the composition food or kit described herein and another food composition, as well as (iii) in animals fed with the said food composition or kit as a supplement food or a drug.

In certain embodiments, (ii) “partly” means that the said animal food composition or kit represents at least about 30% of the animal meal, at least about 50% of said animal meal, or even at least about 70% of said animal meal, or even at least about 80% of said animal meal, or even at least about 90% of said animal meal.

In certain embodiments, the food composition or the kit described herein is provided to animals, especially to dogs, on a daily basis.

In certain embodiments, an animal food composition or kit as disclosed herein can be provided to the animal to be treated as the sole nutritionally complete food during the time period of treatment. According to these embodiments, the said nutritionally complete animal food composition or kit is provided to the animal on a daily basis during the time period of treatment.

In certain other embodiments, an animal food composition or kit as described herein can be provided to the animal to be treated in alternation with an other animal food composition. In certain embodiments, the other animal food composition is a nutritionally complete animal food composition, which other nutritionally complete food composition can be selected among known animal food compositions, including the large variety of commercialized animal food compositions, such as the large variety of commercialized dog food compositions.

In certain embodiments, the said animal food composition or kit is provided each alternate day, thus according to a time schedule of every two days. In another aspect of these embodiments, the said animal food composition or kit is provided according to a time schedule of every three, four, five, six or seven days.

It shall be understood that, in the daily practice of feeding animals, especially dogs, the animal owner cannot proceed according to a systemic way of feeding the animal with an animal food composition always on daily basis. In certain embodiments, the beneficial effects of preventing or treating allergic inflammatory skin diseases, and particularly of preventing or treating canine allergic dermatitis, are fully provided when the animal is fed with the animal food composition or kit described herein once every few days. In particular embodiments, feeding the animal every three, four, five, six or seven days will cause a reduced beneficial effect, which can require a longer time period of treatment.

Without wishing to be bound by any particular theory, it is hypothesized that an efficient prevention or treatment of allergic inflammatory skin diseases, such as a fully efficient prevention or treatment of canine allergic dermatitis requires that the animal, especially the dog, can be provided with an animal food composition or kit as described herein at least, if the food composition or the kit is provided to the animal once every few days, such as e.g., every other, every three, every four, every five, every six or every seven days. In certain particular embodiments, the food composition or the kit is provided to the animal on a daily basis.

The time period of feeding with an animal food composition or kit as described herein can range from several weeks to several years, depending notably on the severity of the allergic inflammatory skin diseases, such as atopic dermatitis.

As already specified elsewhere in the present specification, in certain embodiments, a short time period, e.g., 1 month, of treatment can be sufficient to decrease pruritus.

In certain other embodiments, the animal food composition or kit is provided to the animals to be treated for a longer period of time, such as for about 9 months or more, or about 12 months or more, either (i) according to a feeding schedule comprising providing to the animal, especially a dog, exclusively the animal food composition or kit described herein or (ii) according to a schedule alternating the animal food composition or kit described herein and another animal food composition.

The present disclosure also relates to a method for preventing and/or treating allergic inflammatory skin diseases in an animal affected with allergic inflammatory skin disease including at least a step of making a feeding recommendation that includes an animal food composition or kit as defined in the present disclosure.

The present disclosure further relates to the use of a combination of a source of glycyrrhizin, and of a source of one or more curcuminoids; for the preparation of a composition for the treatment of allergic inflammatory skin diseases.

The present disclosure further relates to the use of a kit comprising a first part comprising a source of glycyrrhizin, and a second part comprising a source of one or more curcuminoids; for the preparation of a composition for the treatment of allergic inflammatory skin diseases.

### Examples

The presently disclosed subject matter will be better understood by reference to the following Examples, which are provided as exemplary of the disclosure, and not by way of limitation. The materials and methods used in the Examples are summarized below.

### A. Materials and Methods

A.1. Objective

The purpose of this clinical trial was to evaluate the impact of a novel animal food composition and the time on PVAS, Drug Score and CADESI-04 and to estimate the actual power at 3, 6 and 9-month time point for the 3 outcomes.

A.2. Material and methods

Dogs

Privately owned atopic dogs with non-seasonal pruritus were included. The diagnosis of atopic dermatitis was made using standard criteria, after exclusion of ectoparasites. Dogs with active bacterial and/or yeasts infections as well as individuals responding adequately to an elimination diet, namely, dogs classified as food allergic dogs, were not included. Dogs undergoing allergen-specific immunotherapy for less than 12 months or dogs controlled with this intervention alone were not enrolled. Included dogs should had an ongoing ectoparasites treatment and this treatment was continued throughout the study. Concomitant medications were allowed throughout the study to ensure an optimal quality of life but were carefully recorded.

Inclusion Consultation, Scorings, and Method of Measurement

Included dogs were initially evaluated for skin lesions and pruritus using CADESI-04 and Hills' Pruritus Visual Analogue Score (PVAS), respectively (Hill et al., 2007; Olivry et al., 2014).

Medications used during the last month before inclusion were recorded. A score was determined using methods as published previously (Litzlbauer P, et al., 2014; Oral and subcutaneous therapy of canine atopic dermatitis with recombinant feline interferon omega). Briefly, the method consists in associating, monthly each drug, dosage and frequency with a score and the final score is defined by adding all the drug associated scores (see Table 1).

Further, the quality of the fur was assessed by the owners of dogs on a scale ranging from 0 to 10. Owners further assessed the quality of the stools ranging from 1 (liquid) to 5 (very dry).

A total of 40 dogs were included and allocated in one of the two study groups, 20 dogs in the tested group and 20 dogs in the placebo group.

Food

The study was double-blinded and placebo-controlled. Included dogs were randomly allocated in one of the two study groups.

One group of dogs received the tested food (Test) while the other group received a premium food (placebo: Control) from the same company, with the same basic ingredients.

As mentioned above neither the owners nor the investigators knew whether a particular dog received a test composition or a placebo composition. Owners were instructed to feed their dogs only with the provided food during the whole study. An informed consent form was signed. The protocol was approved by the local ethical committee.

TABLE 2
Compositions tested

| INGREDIENTS- | CONTROL FOOD | | TEST FOOD | |
|---|---|---|---|---|
| Proximates | % DM | g/Mcal | % DM | g/Mcal |
| Protein | 25 | 61.16 | 25 | 61.25 |
| Crude fat | 15 | 38.01 | 15 | 37.96 |
| Ash | 8 | 18.72 | 8 | 18.75 |
| Crude fibre | 3 | 7.88 | 3 | 7.85 |
| NFE | 49 | 120.13 | 49 | 120.12 |
| Taurine | 0.064 | 0.15 | 0.5 | 1.2 |
| Lutein | 0.00008 | 0.0002 | 0.0005 | 0.0013 |
| EPA/DHA | 0.00001 | 0.02 | 0.6 | 1.5 |
| Linoleic acid | 2 | 5.9 | 4.27 | 10.5 |
| Curcuminoids | 0 | 0 | 0.028 | 0.068 |
| Glycyrrhizin | 0 | 0 | 0.022 | 0.054 |
| Vitamin E | 0.0179 | 0.043 | 0.1 | 0.244 |

“% DM”: Weight percent, based on the total weight of dry matter of the food composition
“g/Mcal”: Metabolizable energy density, as expressed as g/Mcal
“NFE”: Nitrogen Free Extract
“Starch”: The starch content is comprised in the NFE content of the food compositions

Follow-Up Consultations and Drop-Out

Follow-up consultations were made after 1-month (phone call), 3, 6 and 9 months. The first consultation (at 1 month) aimed at recording any problems associated with the study and recording the Medication Score and PVAS. Consultations after 3, 6 and 9 months consisted in general examination of the dogs, CADESI-04, PVAS, Drug Score (Medication Score), stool and fur quality scorings.

Dogs could drop out of the study at any time and for any reason. However, owners were encouraged to feed their dogs with the same food for at least three months. Owners unsatisfied with the efficacy of the provided food were offered to have their pet on the tested food for six additional months, using another name than the two initial foods to guarantee the blinding. Drop-out reasons were recorded. Dogs maintained on the foods for at least three months were taken into account for the analyses. For patients not completing the whole study, the last available data were used for the analyses (last data carry forward procedure).

Outcomes Measures

CADESI-04, PVAS and Medication Scores were recorded for each dog at inclusion and at the end of the study, or in case of early drop-out during the last control consultation. Mean scores of both groups were compared using stool and fur quality scores analyzed qualitatively. Statistical analyzes were carried out using Graphpad Prism 7software (La Jola, Calif., US). Drop-out reasons and unexpected events were recorded.

Comparisons of Means

After checking the normal distribution of the data, parametric methods statistical methods were used. Mean CADESI-04, PVAS and Medication Score at inclusion were first controlled to assess that groups were not statistically significantly different. CADESI-04, PVAS and Medication Scores of both groups (Test versus Control) were compared at study completion, using t test. Finally, CADESI-04, PVAS and Medication Scores were compared within groups (inclusion vs completion) using paired t test.

Proportion of Dogs with 50% Reduction or Returning to Baseline

For each score the proportion of dogs with at least 50% improvement was assessed in each group. Additionally, the proportion of dogs returning to normal, namely with a CADESI-04, PVAS and Medication Scores below 12, 2.5 and 5 respectively were assessed in both groups.

Evaluation of the Overall Response

The overall response to treatment was considered excellent when all 3 outcome scores decreased at the same time by more than 50%, good when two of these 3 outcome scores decrease by more than 50% while the other score remained stable. The proportion of dogs in these assessment groups was computed for each food.

### B. Results

B.1. Individual Comparisons

After checking the normal distribution of the data, parametric methods have been used. Mean CADESI-04, PVAS and Medication Score at inclusion were first controlled to assess that groups were not statistically significantly different. CADESI-04, PVAS and Medication Scores of both groups (Test versus Control) were compared at study completion, using t test. Finally, CADESI-04, PVAS and Medication Score were compared within groups (inclusion vs completion) using matched pair t test (P<0.05).

Actual values for measures are shown in Tables 2a and 2b.

TABLE 2a

| | | | 1 | 3 | 6 | 9 |
|---|---|---|---|---|---|---|
| TEST | | Initial | month | month | month | month |
| CADESI-04 | Mean | 24.3 | | 15.5 | 19.3 | 14.3 |
| | Median | 19 | | 12 | 6.5 | 8 |
| PVAS | Mean | 5.5 | 4.5 | 3.1 | 3.8 | 2.5 |
| | Median | 5.5 | 4.2 | 3 | 2.1 | 2.8 |
| DRUG | Mean | 19.5 | 19.2 | 12.7 | 10.8 | 6.4 |
| | Median | 20 | 20 | 10 | 5 | 5 |

TABLE 2b

| | | | 1 | 3 | 6 | 9 |
|---|---|---|---|---|---|---|
| CONTROL | | Initial | month | month | month | month |
| CADESI-04 | Mean | 23.5 | | 17.6 | 27.2 | 15.3 |
| | Median | 16 | | 13.5 | 5.3 | 7 |
| PVAS | Mean | 4.7 | 4.7 | 4.2 | 4.2 | 3.2 |
| | Median | 4.5 | 4.8 | 4.1 | 2.7 | 2 |
| DRUG | Mean | 24.4 | 26.5 | 26.1 | 25 | 10 |
| | Median | 25 | 30 | 25 | 11.2 | 10 |

Table 3 provides the statistical significance shown for individual comparisons within each diet group (Test paired and Control paired) and comparisons at each time-point between the diet groups (Test versus Control).

TABLE 3

| | | Test | Control | Test vs |
|---|---|---|---|---|
| Timepoint | Score | paired | paired | Control |
| Month 1 | PVAS | 0.02 | 0.33 | 0.41 |
| | DRUG | 0.18 | 0.33 | 0.07 |
| Month 3 | CADESI | 0.0005 | 0.09 | 0.39 |
| | PVAS | <0.0001 | 0.29 | 0.09 |
| | DRUG | 0.006 | 0.22 | 0.006 |
| Month 6 | CADESI | 0.06 | 0.33 | 0.29 |
| | PVAS | 0.01 | 0.24 | 0.001 |
| | DRUG | 0.007 | 0.3 | 0.003 |
| Month 9 | CADESI | 0.004 | 0.28 | 0.49 |
| | PVAS | 0.008 | 0.19 | 0.49 |
| | DRUG | 0.01 | 0.06 | 0.21 |

Forty dogs were initially included. The numbers of dogs involved at each check point are as follows:
1 month: 36 dogs (19 Test, 17 Control)
3 months: 31 dogs (17 Test, 14 Control)
6 months: 24 dogs (12 Test, 12 Control)
9 months: 21 dogs (9 Test, 12 Control)

Initially the mean CADESI-04 in the Test and Control groups were 24.3 and 23.5, p: 0.83. The mean PVAS were 5.5 and 4.7, respectively p: 0.27. The DRUG scores were 19.5 and 24.4, respectively, p: 0.63. Both groups were consequently considered equivalents.

As far a CADESI-04 is concerned, at study completion (month 9) mean CADESI-04 was 15.3 and 14.3 in the Control and Test group, p. 0.35. The reduction was however significant in the Test group (24.3 to 14.3, p: 0.004) but not in the Control Group (23.5 to 15.3, p: 0.28).

Regarding PVAS, comparison between groups at study completion (month 9) was not statistically significant (P. 0.49) but pairwise analysis in the Test group was highly significant (reduction of mean pruritus from 5.5 to 2.5, p: 0.008) but not in the Control group (4.7 to 3.2, p: 0.19).

Finally, DRUG scores were compared, and comparison of both groups were also not significant at study completion (month 9) (p:0.21) but within group a similar trend was visible. In fact, in the Test group the reduction was from 19.5 to 6.4 (p. 0.01) while in the Control group the reduction was more limited (24.4 to 10, p: 0.06).

In the Control Group a 50% improvement of the CADESI-04, PVAS and Medication Score was seen in 3, 1 and no dogs while in the Test group these figures were 8, 7 and 6 (53%, 47% and 40%).

As far as the overall response is concerned, only 1 of the dogs in the Control group was considered good (none excellent) while, in the Test group 5 were excellent and 2 additional were good, representing 47% of excellent or good responses.

Finally, it is noticeable that 6 dogs returned to normal in the 3 outcome scores in the Test group while none corresponded to this definition in the Control group.

In summary, the statistical analyses show that the Test diet had a significant impact on all 3 outcome measures versus time zero at each time-point from 3 months onwards. The Control diet had no effect at any time point versus time zero. The Test diet also had a significant effect on PVAS within the first month. In addition, there was a significant difference between the diet groups for drug score at 3 and 6 months, and for PVAS (pruritus) at 6 months.

B.2. Linear Mixed Models for all Measures

Linear mixed models were used for evaluating the impact of Diet, Time and Time by Diet interactions on CADESI-04, PVAS and Drug Score. Animal was modelled as random term.

Log transformation was used when necessary to meet the statistical assumptions of Linear Mixed Model (normally distributed residuals and homoscedasticity). Tukey HSD was used for the post-hoc multiple comparisons. Level of significance was set at 5%.

Statistical Outcomes

CADESI-04:

With timepoint 9 data excluded, M03 Test group was less than M00 Test group at significance P=0.066.

PVAS (Pruritus):

With timepoint 9 data included, M03 Test group was less than M00 Test group (P=0.0001).

With timepoint 9 data included, M09 Test group was less than M00 Test group (P=0.0043).

With timepoint 9 data excluded, M03 Test group was less than M00 Test group (P=0.0001).

DRUG SCORE (Medication Score):

With timepoint 9 data included, M06 Test group was less than M00 Test group (P=0.021).

With timepoint 9 data included, M09 Test group was less than M00 Test group (P=0.018).

With timepoint 9 data included, M03 Test group was less than M00 Test group (P=0.058).

With timepoint 9 data included, M09 Test group was less than M01 Test group (P=0.039).

With timepoint 9 data included, M06 Test group was less than M01 Test group (P=0.052).

With timepoint 9 data excluded, M06 Test group was less than M00 Test group (P=0.03).

B.3. Combined Score

CADESI-04, PVAS and Drug Score were combined in a single score variable. The combined score was calculated by averaging the three different outcome scores after a standardization process (mean centering and scaling).

Statistical Outcomes

With timepoint 1 data excluded, timepoint 9 data included, M03 Test group was less than M00 Test group (P=0.0008).

With timepoint 1 data excluded, timepoint 9 data included, M09 Test group was less than M00 Test group (P=0.0052).

With both timepoint 1 and timepoint 9 data excluded, M03 Test group was less than M00 Test group (P=0.0014).

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

For any patents, patent applications, publications, product descriptions, and protocols are cited throughout this application, the disclosures of all of which are incorporated herein by reference in their entireties for all purposes.

1-9. (canceled)

10. An animal food composition comprising:
(i) a source of glycyrrhizin, in combination with
(ii) a source of one or more curcuminoids;
wherein the animal food composition consists of a kibble and
wherein the animal food composition optionally comprises a source of proteins.

11. The animal food composition according to claim 10, wherein the animal food composition consists of a nutritionally complete food composition.

12. The animal food composition of claim 10, wherein:
(i) the source of glycyrrhizin consists of licorice, and/or
(ii) the source of one or more curcuminoids consists of turmeric.

13. The animal food composition of claim 10, further comprising linoleic acid.

14. The animal food composition of claim 10, further comprising one or more of EPA/DHA, Taurine, Lutein, Vitamin E or a combination thereof.

15. A method of manufacturing an animal food composition of claim 10, comprising the steps of:
a) mixing a source of a glycyrrhizin and a source of one or more curcuminoid, thereby providing a mixture; and
b) heating the mixture.

16. A method for preventing and/or treating an allergic inflammatory skin disease in an animal in need thereof, comprising administering an animal food composition comprising:
(i) a source of glycyrrhizin, and
(ii) a source of one or more curcuminoids.

17. The method of claim 16, wherein the one or more curcuminoids is a compound of general formula (I), a pharmaceutically acceptable salt, a tautomer, or a stereoisomer thereof: 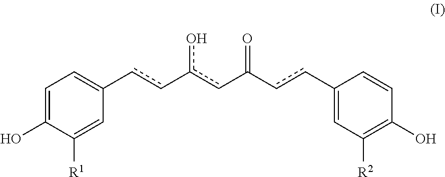
wherein R¹ and R² are independently hydrogen atom methoxy, methyl, hydroxyl or ethoxy group.

18. The method of claim 16, wherein the one or more curcuminoids are selected from the group consisting of curcumin, demethoxycurcumin, methoxycurcumin, tetrahydrocurcumin, and combinations thereof.

19. The method of claim 16, wherein the source of glycyrrhizin is licorice.

20. The method of claim 16, wherein the animal food composition comprises licorice and turmeric.

21. The method of claim 16, wherein the allergic inflammatory skin disease is selected from the group consisting of atopic dermatitis, flea allergic dermatitis, urticaria, angioedema, inhalant allergy, inhalant allergic dermatitis, food allergic dermatitis, contact dermatitis, miliary dermatitis, eosinophilic granuloma, head and neck pruritus, and generalized pruritus.

22. The method of claim 16, wherein the animal food composition further comprises linoleic acid, EPA/DHA, Taurine, Lutein, Vitamin E or combinations thereof.

23. The method of claim 16, wherein the animal is a canine.

24. The method of claim 23, wherein the canine is a dog.
